(19) United States
(12) Patent Application Publication
(10) Pub. No.: US 2019/0049447 A1
(43) Pub. Date: Feb. 14, 2019

(54) METHODS AND COMPOSITIONS FOR DETECTING COCCIDIOIDOMYCOSIS

(71) Applicant: ARIZONA BOARD OF REGENTS ON BEHALF OF THE UNIVERSITY OF ARIZONA, Tucson, AZ (US)

(72) Inventors: John N. Galgiani, Tucson, AZ (US); Michael D. L. Johnson, Tucson, AZ (US)

(21) Appl. No.: 16/058,538

(22) Filed: Aug. 8, 2018

Related U.S. Application Data
(60) Provisional application No. 62/542,594, filed on Aug. 8, 2017.

(51) Int. Cl. G01N 33/573; B01L 3/00; C12N 9/42

(52) U.S. Cl. CPC G01N 33/573; B01L 3/508; G01N 2333/942; C12Y 302/01014; C12N 9/2442

(57) ABSTRACT

Methods and compositions for detecting Coccidioidomycosis featuring amino acids 105-310 of the Cts1 protein or amino acids 111-310 of the Cts1 protein; or peptide sequences that are similar to amino acids 105-310 of the Cts1 protein or similar to amino acids 111-310 of the Cts1 protein. Compared to standard immunodiffusion testing of undiluted sera for these antibodies, the present invention shows specific detection above non-immune sera at 1:100 dilution. The methods and compositions of the present invention help reduce the non-specific antibody binding unrelated to acquiring a coccidioidal infection, thereby providing a more sensitive test for early coccidioidal infection because signal could be distinguished at lower intensity, avoiding confusion with nonspecific antibody binding.

### CROSS REFERENCE

This application claims priority to U.S. Provisional Patent Application No. 62/542,594 filed Aug. 8, 2017, the specification(s) of which is/are incorporated herein in their entirety by reference.

### GOVERNMENT SUPPORT

This invention was made with government support under Grant No. R01 AI132140 awarded by NIH. The government has certain rights in the invention.

### REFERENCE TO SEQUENCE LISTING

Applicant asserts that the paper copy of the Sequence Listing is identical to the Sequence Listing in computer readable form found on the accompanying computer file, entitled >>>UNIA_17_33_NP_ST25.txt<<<. The content of the sequence listing is incorporated herein by reference in its entirety.

### FIELD OF THE INVENTION

The present invention relates to the detection of coccidioidomycosis, more particularly to methods and anti-coccidioidal complement fixing antibodies specific for certain truncations of chitinase.

### BACKGROUND OF THE INVENTION

The detection of anti-coccidioidal complement fixing (CF) antibodies in serum, cerebrospinal fluid, and other bodily fluids has been the basis for highly specific diagnostic tests and its quantitation has prognostic value. Past studies from three independent laboratories have identified the chitinase enzyme, expressed from cts1, as the 427 amino acid protein (Cts1) that CF antibodies recognize. In a previous analysis (see Yang et al., 1997, Infection and Immunity 65(10):4068-4074), an amino acid (a.a.) 20-310 truncation bound CF serum antibodies from patients with coccidioidomycosis but did not cross react with serum from patients with either histoplasmosis or blastomycosis.

Inventors surprisingly found that all or most of the antibody binding (CF antibody binding) is directed toward amino acids 105-310. Inventors also surprisingly discovered that the Coccidioides chitinase gene misses 9 amino acids after amino acid 176 and 12 amino acids after amino acid 260.

Compared to standard immunodiffusion testing of undiluted sera for these antibodies, the present invention shows specific detection above non-immune sera at 1:100 dilution (at least a two-log increase in sensitivity for the same antibodies). The methods and compositions of the present invention will help reduce the non-specific antibody binding unrelated to acquiring a coccidioidal infection. This could provide a more sensitive test for early coccidioidal infection because signal could be distinguished at lower intensity, avoiding confusion with nonspecific antibody binding.

### SUMMARY OF THE INVENTION

The present invention features methods of detecting coccidioidomycosis. In some embodiments, the method comprises subjecting a test sample to an enzyme conjugate assay wherein the sample is subjected to a peptide comprising amino acids 105-310 of Cts1. In some embodiments, the method provides a proportional result as compared to CF titrations. In some embodiments, the method determines the absorbance threshold at which the CF titer would correspond to a specific specimen dilution.

The present invention provides methods for detecting coccidioidomycosis. In some embodiments, the method comprises contacting a test sample with a Cts1 peptide according to SEQ ID NO: 4; and making binding of an antibody in the test sample to the Cts1 peptide detectable, e.g., using detection reagents or schemes to make the binding of antibodies in the test sample to the Cts1 peptide visible. Detection of binding of the Cts1 peptide to antibodies in the test sample may be indicative of coccidioidomycosis. In some embodiments, the Cts1 peptide is attached to a solid surface. In some embodiments, the method is an enzyme conjugate assay. In some embodiments, the method is an enzyme-linked immunosorbent assay (ELISA). In some embodiments, the method is a complement fixation assay.

The present invention also provides an isolated Cts1 peptide having a sequence that is at least 90% identical to SEQ ID NO: 4. In some embodiments, the Cts1 peptide has a sequence that is at least 95% identical to SEQ ID NO: 4. In some embodiments, the Cts1 peptide has a sequence that is at least 99% identical to SEQ ID NO: 4. In some embodiments, the Cts1 peptide is attached to a solid surface. In some embodiments, the Cts1 peptide is covalently bound to a solid surface.

The present invention also provides assay platforms (or kits) for detecting coccidioidomycosis. In some embodiments, the platform comprises a solid support; and an isolated Cts1 peptide having a sequence according to SEQ ID NO: 4 (or a sequence that is at least 90% identical, at least 95% identical, at least 98% identical at least 99% identical to SEQ ID NO: 4) attached to the solid support. In some embodiments, the assay platform is for ELISAs. In some embodiments, the assay platform is for a complement fixation assay. In some embodiments, the solid support is a well. In some embodiments, the well is part of a microwell plate. In some embodiments, the isolated Cts1 peptide is covalently attached to the solid support.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

This patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows SDS PAGE of E. coli-expressed rCTS1 truncations after Ni-NTA affinity purification.

FIG. 2 shows an immunoblot of E. coli-expressed rCTS1 truncations with CF+human serum.

FIG. 3 shows that all CF antibodies bind CTS1 111-310.

FIG. 4 shows ELISA and CF activities are comparable. Briefly, microtiter plates were prepared using with 100 ng of CTS1 111-310 per well as described above. Human sera with CF titers from undetectable to 1:64 were diluted 1:100 and then proportionally to the CF titer. For example sera of CF titer of 1:1 were diluted 1:100 and sera of CF titer of 1:64 were diluted 1:6400. Blocking prior to sera application, and enzyme conjugate detection of antibodies and absorbance measurements were conducted as described above.

FIG. 5 shows an ELISA assay featuring four subunits of the CTS105-310 truncation and CTS105-310. The figure shows evidence of no binding for four subunits of Cts1, whereas CTS105-310 has complete binding. This may be a prototype for a diagnostic test procedure.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety herein by reference.

All publications, patent applications, patents, and other references mentioned herein are incorporated by reference in their entirety.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Sequence identity: The identity (or similarity) between two or more nucleic acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN may be used to compare nucleic acid sequences, while BLASTP may be used to compare amino acid sequences. If the two compared sequences share homology, then the designated output file will present those regions of homology as aligned sequences. If the two compared sequences do not share homology, then the designated output file will not present aligned sequences. The BLAST-like alignment tool (BLAT) may also be used to compare nucleic acid sequences (Kent, Genome Res. 12:656-664, 2002). BLAT is available from several sources, including Kent Informatics (Santa Cruz, Calif.) and on the Internet (genome.ucsc.edu). Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth.

Sample: Any composition containing or presumed to contain a biomarker or a composition being tested for the presence or absence of a particular biomarker or other biological entity such as an antibody or fragment thereof. Samples may include purified or separated components of cells, tissues, or blood, e.g., DNA, RNA, proteins, cell-free portions, or cell lysates. The sample can also be from a fresh liquid sample or a previously frozen sample. In certain embodiments, the sample is a liquid sample, e.g., blood or a blood component (plasma or serum), urine, semen, saliva, sputum, mucus, semen, tear, lymph, cerebral spinal fluid, material washed from a swab, etc. The sample can also be partially processed from a sample directly obtained from an individual.

### DETAILED DESCRIPTION OF THE INVENTION

The present invention features methods and compositions for detecting coccidioidomycosis. Inventors surprisingly found that all or most of the antibody binding (CF antibody binding) is directed toward amino acids 105-310 or amino acids 111-310. The methods of the present invention allow for a specific detection above non-immune sera at a 1:100 dilution (an increase in sensitivity). The methods and compositions of the present invention will help reduce the non-specific antibody binding unrelated to acquiring a coccidioidal infection. This could provide a more sensitive test for early coccidioidal infection because signal could be distinguished at lower intensity, avoiding confusion with nonspecific antibody binding.

For reference, the sequence for chitinase (SEQ ID NO: 1; Coccidioides posadasii, GenBank: AAA92643.1) is shown below in Table 1.

TABLE 1

| SEQ | | |
|---|---|---|
| ID | | |
| NO: | DESCRIPTION | SEQUENCE |
| 1 | Chitinase | MRFLIGALLT LQTLVQASSM SSMPNYYPVP EAPAEGGFRS |
| | (Cts1) | VVYFVNWAIY GRGHNPQDLK ADQFTHILYA FANIRPSGEV |
| | | YLSDTWADTD KHYPGDKWDE PGNNVYGCIK QMYLLKKNNR |
| | | NLKTLLSIGG WTYSPNFKTP ASTEEGRKKF ADTSLKLMKD |
| | | LGFDGIDIDW EYPEDEKQAN DFVLLLKACR EALDAYSAKH |
| | | PNGKKFLLTI ASPAGPQNYN KLKLAEMDKY LDFWNLMAYD |
| | | FSGSWDKVSG HMSNVFPSTT KPESTPFSSD KAVKDYIKAG |
| | | VPANKIVLGM PLYGRAFAST DGIGTSFNGV GGGSWENGVW |
| | | DYKDMPQQGA QVTELEDIAA SYSYDKNKRY LISYDTVKIA |
| | | GKKAEYITKN GMGGGMWWES SSDKTGNESL VGTVVNGLGG |
| | | TGKLEQRENE LSYPESVYDN LKNGMPS |

Testing of Truncated Peptides Cts1₂₀₋₃₁₀ and Cts1₁₁₁₋₃₁₀

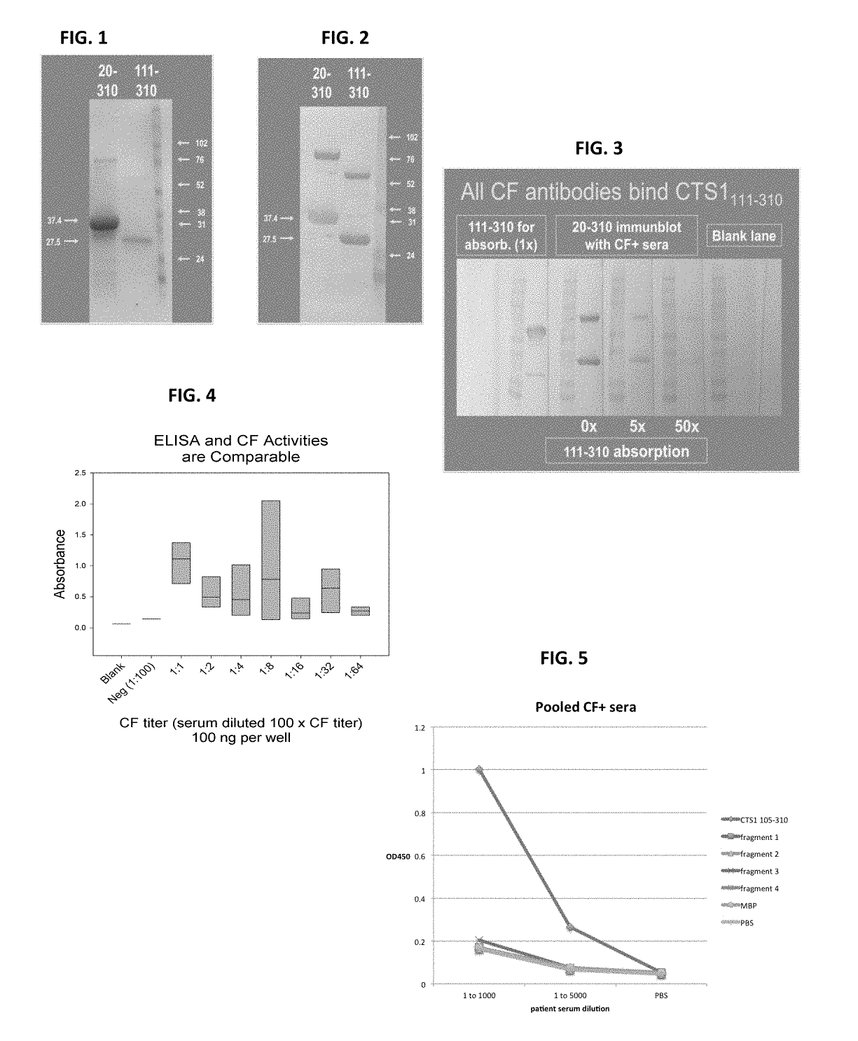

Amplimers of the full-length cts1 and truncations encoding a.a.20-310 (Cts1₂₀₋₃₁₀) (SEQ ID NO: 2, see Table 2) and a.a.111-310 (Cts1₁₁₁₋₃₁₀) (SEQ ID NO: 3, see Table 2) were generated by PCR with DNA encoding a poly-His sequence added to the 3′ end. Amplimers were cloned into pMCSG7 for transfection into E. coli BL21(DE₃) for expression. Recombinant peptides were extracted with urea (e.g., 7M, 8M), and preliminary purification was accomplished by affinity binding to a nickel-NTA column. Following renaturation (e.g., dialyze to PBS), proteins were analyzed by PAGE and subsequent immunoblotting. For example, FIG. 1 shows SDS PAGE of E. coli-expressed rCTS1 truncations after Ni-NTA affinity purification. FIG. 2 shows an immunoblot of E. coli-expressed rCTS1 truncations with CF+human serum.

TABLE 2

| SEQ | | |
|---|---|---|
| ID | | |
| NO: | DESCRIPTION | SEQUENCE |
| 2 | Cts1₂₀₋₃₁₀ | M SSMPNYYPVP EAPAEGGFRS VVYFVNWAIY GRGHNPQDLK |
| | | ADQFTHILYA FANIRPSGEV YLSDTWADTD KHYPGDKWDE |
| | | PGNNVYGCIK QMYLLKKNNR NLKTLLSIGG WTYSPNFKTP |
| | | ASTEEGRKKF ADTSLKLMKD LGFDGIDIDW EYPEDEKQAN |
| | | DFVLLLKACR EALDAYSAKH PNGKKFLLTI ASPAGPQNYN |
| | | KLKLAEMDKY LDFWNLMAYD FSGSWDKVSG HMSNVFPSTT |
| | | KPESTPFSSD KAVKDYIKAG VPANKIVLGM PLYGRAFAST |
| | | DGIGTSFNGV |
| 3 | Cts1₁₁₁₋₃₁₀ | QMYLLKKNNR NLKTLLSIGG WTYSPNFKTP ASTEEGRKKF |
| | | ADTSLKLMKD LGFDGIDIDW EYPEDEKQAN DFVLLLKACR |
| | | EALDAYSAKH PNGKKFLLTI ASPAGPQNYN KLKLAEMDKY |
| | | LDFWNLMAYD FSGSWDKVSG HMSNVFPSTT KPESTPFSSD |
| | | KAVKDYIKAG VPANKIVLGM PLYGRAFAST DGIGTSFNGV |

Immunoblots demonstrated that both the a.a.20-310 (Cts1₂₀₋₃₁₀; SEQ ID NO: 2) and the a.a.111-310 (Cts1₁₁₁₋₃₁₀; SEQ ID NO: 3) recombinant peptides reacted to sera from patients with coccidioidomycosis that contained CF antibodies but did not react with sera from uninfected patients. Absorption of CF positive sera with a.a.111-310 (Cts1₁₁₁₋₃₁₀; SEQ ID NO: 3) eliminated immunoblot binding of the sera to a.a.20-310 (Cts1₂₀₋₃₁₀; SEQ ID NO: 2) (data not shown).

The present invention describes isolated peptides of Cts1 that retain affinity for CF antibodies. These results help provide the basis for a reference enzyme-linked immunoassay to mimic quantitative results currently produced by the originally described CF antibody detection assay. FIG. 4 demonstrates that absorbance resulting from enzyme conjugate detection of antibodies is proportional to the CF titers. Therefore, measurement of absorbance of several different dilutions of a patient serum or other bodily fluid will result in absorbance curves that are proportional to the amount of CF antibody activity in the specimen. Using specimens of known CF antibody titers, a final protocol could determine the absorbance threshold at which the CF titer would correspond to a specific specimen dilution.

The present invention provides an isolated peptide according to SEQ ID NO: 3. The present invention also feature peptides that are similar to SEQ ID NO: 3, e.g., peptides wherein one amino acid is different, two amino acids are different, three amino acids are different, four amino acids are different, five amino acids are different, six amino acids are different, seven amino acids are different, eight amino acids are different, nine amino acids are different, ten amino acids are different, more than 10 amino acids are different, more than 20 amino acids are different, more than 30 amino acids are different, 20-30 amino acids are different, 1-10 amino acids are different, 10-20 amino acids are different, 30-40 amino acids are different, 40-50 amino acids are different, etc. Stated differently, in some embodiments, the peptide is at least 75% identical to SEQ ID NO: 3. In some embodiments, the peptide is at least 80% identical to SEQ ID NO: 3. In some embodiments, the peptide is at least 85% identical to SEQ ID NO: 3. In some embodiments, the peptide is at least 90% identical to SEQ ID NO: 3. In some embodiments, the peptide is at least 95% identical to SEQ ID NO: 3. In some embodiments, the peptide is at least 96% identical to SEQ ID NO: 3. In some embodiments, the peptide is at least 97% identical to SEQ ID NO: 3. In some embodiments, the peptide is at least 98% identical to SEQ ID NO: 3. In some embodiments, the peptide is at least 99% identical to SEQ ID NO: 3. In some embodiments, the peptide is at least 100% identical to SEQ ID NO: 3.

### Testing of Truncated Peptide Cts1₁₀₅₋₃₁₀

Referring to FIG. 5, a CTS1 truncation (Cts1₁₀₅₋₃₁₀; SEQ ID NO: 4, see Table 3) as well as four subunits of Cts1₁₀₅₋₃₁₀ (SEQ ID NO: 4) were tested for binding in an ELISA. FIG. 4 shows complete binding of Cts1₁₀₅₋₃₁₀ (SEQ ID NO: 4) but essentially no binding of the subunits. Without wishing to limit the present invention to any theory or mechanism, it is believed that a similar assay may be used as a diagnostic test procedure.

The present invention provides an isolated peptide according to SEQ ID NO: 4. The present invention also feature peptides that are similar to SEQ ID NO: 4, e.g., peptides wherein one amino acid is different, two amino acids are different, three amino acids are different, four amino acids are different, five amino acids are different, six amino acids are different, seven amino acids are different, eight amino acids are different, nine amino acids are different, ten amino acids are different, more than 10 amino acids are different, more than 20 amino acids are different, more than 30 amino acids are different, 20-30 amino acids are different, 1-10 amino acids are different, 10-20 amino acids are different, 30-40 amino acids are different, 40-50 amino acids are different, etc. Stated differently, in some embodiments, the peptide is at least 75% identical to SEQ ID NO: 4. In some embodiments, the peptide is at least 80% identical to SEQ ID NO: 4. In some embodiments, the peptide is at least 85% identical to SEQ ID NO: 4. In some embodiments, the peptide is at least 90% identical to SEQ ID NO: 4. In some embodiments, the peptide is at least 95% identical to SEQ ID NO: 4. In some embodiments, the peptide is at least 96% identical to SEQ ID NO: 4. In some embodiments, the peptide is at least 97% identical to SEQ ID NO: 4. In some embodiments, the peptide is at least 98% identical to SEQ ID NO: 4. In some embodiments, the peptide is at least 99% identical to SEQ ID NO: 4. In some embodiments, the peptide is at least 100% identical to SEQ ID NO: 4. Non-limiting examples of peptides that are similar to SEQ ID NO: 4 are shown in Table 3 below.

TABLE 3

| SEQ | | |
|---|---|---|
| ID | | |
| NO: | DESCRIPTION | SEQUENCE |
| 4 | Cts1₁₀₅₋₃₁₀ | VYGCIK QMYLLKKNNR NLKTLLSIGG WTYSPNFKTP |
| | | ASTEEGRKKF ADTSLKLMKD LGFDGIDIDW EYPEDEKQAN |
| | | DFVLLLKACR EALDAYSAKH PNGKKFLLTI ASPAGPQNYN |
| | | KLKLAEMDKY LDFWNLMAYD FSGSWDKVSG HMSNVFPSTT |
| | | KPESTPFSSD KAVKDYIKAG VPANKIVLGM PLYGRAFAST |
| | | DGIGTSFNGV |
| 5 | Modified Cts1₁₀₅₋₃₁₀ | VYGCIK QMYLLKKNNR NLKTLLSIGG WTYSPNFKTP |
| | 99% identical to SEQ | ASTEEGRKKF ADTSLKLMKD LGFDGIDIDW EYPEDEKQAN |
| | ID NO: 4; bold letters | DFVLLLKACR EAVDAYSAKH PNGKKFLVTI ASPAGPQNYN |
| | are substituted amino | KLKLAEMDKY LDFWNLMAYD FSGSWDKVSG HMSNVFPSTT |
| | acids | KPESTPFSSD KAVKDYIKAG VPANKIVLGM PLYGRAFAST |
| | | DGIGTSFNGV |
| 6 | Modified Cts1₁₀₅₋₃₁₀ | VYGCIK QMYLLKKNNR NLKTLLSIGG WTYSPNFKTP |
| | 99% identical to SEQ | ASTEEGRKKF LDTSLKLMKD LGFDGIDIDW EYPDDEKQAN |
| | ID NO: 4; bold letters | DFVLLLKACR EALDAYSAKH PNGKKFLLTI ASPAGPQNYN |
| | are substituted amino | KLKLAEMDKY LDFWNLMAYD FSGSWDKVSG HMSNVFPSTT |
| | acids | KPESTPFSSD KAVKDYIKAG VPANKIVLGM PLYGRAFAST |
| | | DGIGTSFNGV |
| 7 | Modified Cts1₁₀₅₋₃₁₀ | VYGCIK QMYLLKKNNR NLKTLLSIGG WTYSPNFKTP |
| | 98% identical to SEQ | ASTEDGRKKF ADTSLKLMKD LGFDGIDIDW EYPEDEKQAN |
| | ID NO: 4; bold letters | DFVLLLKACR EALDAYSAKH PNGKRFLLTI ASPVGPQNYN |
| | are substituted amino | KLKLAEMDKY LDFWNLMAYD FSGSWDKVSG HMSNVFPSTT |
| | acids | KPESTPFSSD KAVKDYIKAG VPANKIVLGM PLYGRAFAST |
| | | DGIMTSFNGV |
| 8 | Modified Cts1₁₀₅₋₃₁₀ | VYGCIK QMYLLKKNNR NLKTLLSIGG WTYSPNFKTP |
| | 98% identical to SEQ | ASTEEGRKKF IDTSLKLMKD LGFDGIDIDW EYPEDEKQAN |
| | ID NO: 4; bold letters | DFVLLLKACR DALDAYSAKH PNVKKFLLTI ASPAGPQNYN |
| | are substituted amino | KLKLAEMDKY LDFWNLMAYD FSGSWDKVSG HMSNVFPSTT |
| | acids | KPESTPFSSD KAVKDYIKAG VPANKIVLGM PLYLRAFAST |
| | | DGIGTSFNGV |
| 9 | Modified Cts1₁₀₅₋₃₁₀ | VYGCIK QMYLLHKNNR NLKTLLSIVL WTYSPNFKTP |
| | 95% identical to SEQ | ISTEEGRKKF ADTSLKLMKD LGFDGIDIDW EYPDDEKQAN |
| | ID NO: 4; bold letters | DFVLLLKACR EALDAYSAKH PNGKRFLLTI ASPAGPQNYN |
| | are substituted amino | KLRLAEMDKY LDFWNLMAYD FSGSWDKVSG HMSNVFPSTT |
| | acids | KPESTPFSSE KAVKDYIKAG VPANKIVLGM PLYGHAFAST |
| | | EGIGTSFNGV |
| 10 | Modified Cts1₁₀₅₋₃₁₀ | VWGCIK QMYLLKKNNR NLKTLLSIGG WTYSPNFKNP |
| | 95% identical to SEQ | ASTEEGRKKF ADTSLKLMKD LGFDGIDIDW EYPEEEKQAN |
| | ID NO: 4; bold letters | DFVMLLKACR EALDAYSVKH PNGKKYLLTI ASPAGPQNYN |
| | are substituted amino | KLKLAEMDKY LDFWNLMAYD FSGSWDKVSG HMSQVFPSTT |
| | acids | KPESTIFSSD KAVKDYIRAG VPANKIVLAM PLYGRAFAST |
| | | DGIGTSFNGV |
| 11 | Modified Cts1₁₀₅₋₃₁₀ | VYGNIK QMYLLRKNNR NLKTLLSIGG WTYSPNFKTA |
| | 90% identical to SEQ | ASNEEGRKKF ADTSLHLMKD LGFEAIDIDW EYPDEDKQAN |
| | ID NO: 4; bold letters | DWVLLLHACR EALDAYSAKH PNGKRFLLTI ASPAGPQNYN |
| | are substituted amino | KLKLAEMDKY LDFWNLMAYD FSGSWDKVSV HMSNVFPSTT |
| | acids | RPESTPFTSD KAVKDYIKAG VPANRVVLGM PLYGRLFAST |
| | | DGIGTTFNGV |
| 12 | Modified Cts1₁₀₅₋₃₁₀ | VYGCIR QMYLLKHNNR NLKTLVSIGG WTYSPNFRTP |
| | 90% identical to SEQ | ASTEEGRKKF ADTSLKLMKD LGFDGIDIDW EYPDEEKQAN |
| | ID NO: 4; bold letters | DFVLLVKACK EVLDAYSAKH PNGHRFLLTI ASPAGPQNYN |
| | are substituted amino | KLKLAEMDKY LDFWNLMIYD FSGSWDKVTG HMSNVFPSTT |
| | acids | RPESTPFTSD KAVKEYIKAG VPLNKIVLGM PLYARAFAST |
| | | DGIVTSFNMV |

The present invention also provides peptide constructs comprising one of the peptides disclosed herein (e.g., SEQ ID NO: 4, SEQ ID NO: 3, etc.) attached to or linked (directly or indirectly) to a component used to bind the peptide to a solid surface. Methods and reagents used for linking or binding a peptide to a solid surface are well known to one of ordinary skill in the art.

The present invention also features nucleic acids that encode any of the peptides disclosed herein (e.g., nucleic acids that encode SEQ ID NO: 3, SEQ ID NO: 4, a peptide similar thereto, etc.).

The present invention also provides expression vectors that can produce any of the peptides disclosed herein.

The present invention also provides assay platforms for detecting coccidioidomycosis. For example, the assay platform may comprise a surface, such as a well, wherein a peptide of the present invention (e.g., SEQ ID NO: 4) is bound to the surface. A sample may be introduced to the assay platform, wherein the sample is contacted with the peptide bound to the surface. Assays such as this may be similar to an ELISA.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method of detecting coccidioidomycosis, said method comprising:
a. contacting a test sample with a Cts1 peptide according to SEQ ID NO: 4;
b. making binding of an antibody in the test sample to the Cts1 peptide detectable;
wherein detecting binding of the Cts1 peptide to an antibody in the test sample is indicative of coccidioidomycosis.

2. The method of claim 1, wherein the Cts1 peptide is attached to a solid surface.

3. The method of claim 1, wherein the method is an enzyme conjugate assay.

4. The method of claim 1, wherein the method is an enzyme-linked immunosorbent assay (ELISA).

5. The method of claim 1, wherein the method is a complement fixation assay.

6. An isolated Cts1 peptide having a sequence that is at least 90% identical to SEQ ID NO: 4.

7. The isolated Cts1 peptide of claim 6, wherein the Cts1 peptide has a sequence that is at least 95% identical to SEQ ID NO: 4.

8. The isolated Cts1 peptide of claim 6, wherein the Cts1 peptide has a sequence that is at least 99% identical to SEQ ID NO: 4.

9. The isolated Cts1 peptide of claim 6, wherein the Cts1 peptide is attached to a solid surface.

10. The isolated Cts1 peptide of claim 6, wherein the Cts1 peptide is covalently bound to a solid surface.

11. An assay platform for detecting coccidioidomycosis, said platform comprising:
a. a solid support; and
b. an isolated Cts1 peptide having a sequence according to SEQ ID NO: 4 attached to the solid support.

12. The platform of claim 11, wherein the assay platform is for ELISAs.

13. The platform of claim 11, wherein the assay platform is for a complement fixation assay.

14. The platform of claim 11, wherein the solid support is a well.

15. The platform of claim 11, wherein the well is part of a microwell plate.

16. The platform of claim 11, wherein the isolated Cts1 peptide is covalently attached to the solid support.


SEQUENCE LISTING (12 sequences)

SEQ ID NO: 1 - protein, 427 aa, Coccidioides immitis
MRFLIGALLTLQTLVQASSMSSMPNYYPVPEAPAEGGFRSVVYFVNWAIYGRGHNPQDLKADQFTHILYAFANIRPSGEVYLSDTWADTDKHYPGDKWDEPGNNVYGCIKQMYLLKKNNRNLKTLLSIGGWTYSPNFKTPASTEEGRKKFADTSLKLMKDLGFDGIDIDWEYPEDEKQANDFVLLLKACREALDAYSAKHPNGKKFLLTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGVGGGSWENGVWDYKDMPQQGAQVTELEDIAASYSYDKNKRYLISYDTVKIAGKKAEYITKNGMGGGMWWESSSDKTGNESLVGTVVNGLGGTGKLEQRENELSYPESVYDNLKNGMPS

SEQ ID NO: 2 - protein, 291 aa, Artificial Sequence
  Amino acids 20-310 of Cts1 (Coccidioides posadiasii, GenBank AAA92643.1)
MSSMPNYYPVPEAPAEGGFRSVVYFVNWAIYGRGHNPQDLKADQFTHILYAFANIRPSGEVYLSDTWADTDKHYPGDKWDEPGNNVYGCIKQMYLLKKNNRNLKTLLSIGGWTYSPNFKTPASTEEGRKKFADTSLKLMKDLGFDGIDIDWEYPEDEKQANDFVLLLKACREALDAYSAKHPNGKKFLLTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGV

SEQ ID NO: 3 - protein, 200 aa, Artificial Sequence
  Amino acids 111-310 of Cts1 (Coccidioides posadiasii, GenBank AAA92643.1)
QMYLLKKNNRNLKTLLSIGGWTYSPNFKTPASTEEGRKKFADTSLKLMKDLGFDGIDIDWEYPEDEKQANDFVLLLKACREALDAYSAKHPNGKKFLLTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGV

SEQ ID NO: 4 - protein, 206 aa, Artificial Sequence
  Amino acids 105-310 of Cts1 (Coccidioides posadiasii, GenBank AAA92643.1)
VYGCIKQMYLLKKNNRNLKTLLSIGGWTYSPNFKTPASTEEGRKKFADTSLKLMKDLGFDGIDIDWEYPEDEKQANDFVLLLKACREALDAYSAKHPNGKKFLLTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGV

SEQ ID NO: 5 - protein, 206 aa, Artificial Sequence
  Amino acids 105-310 of Cts1 (Coccidioides posadiasii, GenBank AAA92643.1), with L193V and L208V substitutions.
VYGCIKQMYLLKKNNRNLKTLLSIGGWTYSPNFKTPASTEEGRKKFADTSLKLMKDLGFDGIDIDWEYPEDEKQANDFVLLLKACREAVDAYSAKHPNGKKFLVTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGV

SEQ ID NO: 6 - protein, 206 aa, Artificial Sequence
  Amino acids 105-310 of Cts1 (Coccidioides posadiasii, GenBank AAA92643.1), with A151L and E174D substitutions.
VYGCIKQMYLLKKNNRNLKTLLSIGGWTYSPNFKTPASTEEGRKKFLDTSLKLMKDLGFDGIDIDWEYPDDEKQANDFVLLLKACREALDAYSAKHPNGKKFLLTIASPAGPQNYNKLKLAEMDKYLDFWNLMAYDFSGSWDKVSGHMSNVFPSTTKPESTPFSSDKAVKDYIKAGVPANKIVLGMPLYGRAFASTDGIGTSFNGV

SEQ ID NO: 7 - protein, 206 aa, Artificial Sequence
  Amino acids 105-310 of Cts1 (Coccidioides posadiasii, GenBank AAA92643.1), with E145D, K205R, A214V, and G304M substitutions.
VYGCIKQMYLLKKNNRNLKTLLSIGGWTYSPNFKTPASTEDGRKKFADTSLKLMKDLGFDGIDIDWEYPEDEKQANDFVLLLKACREALDAYSAKHPNGKRFLLTIASPVGPQNYNKLKLAEMDKYLDFWNLMAYDFSGSWDKVSGHMSNVFPSTTKPESTPFSSDKAVKDYIKAGVPANKIVLGMPLYGRAFASTDGIMTSFNGV

SEQ ID NO: 8 - protein, 206 aa, Artificial Sequence
  Amino acids 105-310 of Cts1 (Coccidioides posadiasii, GenBank AAA92643.1), with A151I, E191D, G203V, and G294L substitutions.
VYGCIKQMYLLKKNNRNLKTLLSIGGWTYSPNFKTPASTEEGRKKFIDTSLKLMKDLGFDGIDIDWEYPEDEKQANDFVLLLKACRDALDAYSAKHPNVKKFLLTIASPAGPQNYNKLKLAEMDKYLDFWNLMAYDFSGSWDKVSGHMSNVFPSTTKPESTPFSSDKAVKDYIKAGVPANKIVLGMPLYLRAFASTDGIGTSFNGV

SEQ ID NO: 9 - protein, 206 aa, Artificial Sequence
  Amino acids 105-310 of Cts1 (Coccidioides posadiasii, GenBank AAA92643.1), with K116H, G129V, G130L, A141I, E174D, K205R, K223R, D270E, R295H,and D301E substitutions.
VYGCIKQMYLLHKNNRNLKTLLSIVLWTYSPNFKTPISTEEGRKKFADTSLKLMKDLGFDGIDIDWEYPDDEKQANDFVLLLKACREALDAYSAKHPNGKRFLLTIASPAGPQNYNKLRLAEMDKYLDFWNLMAYDFSGSWDKVSGHMSNVFPSTTKPESTPFSSEKAVKDYIKAGVPANKIVLGMPLYGHAFASTEGIGTSFNGV

SEQ ID NO: 10 - protein, 206 aa, Artificial Sequence
  Amino acids 105-310 of Cts1 (Coccidioides posadiasii, GenBank AAA92643.1), with Y106W, T139N, D175E, L184M, A198V, F206Y, N2554Q, P266I, K278R, and G289A substitutions.
VWGCIKQMYLLKKNNRNLKTLLSIGGWTYSPNFKNPASTEEGRKKFADTSLKLMKDLGFDGIDIDWEYPEEEKQANDFVMLLKACREALDAYSVKHPNGKKYLLTIASPAGPQNYNKLKLAEMDKYLDFWNLMAYDFSGSWDKVSGHMSQVFPSTTKPESTIFSSDKAVKDYIRAGVPANKIVLAMPLYGRAFASTDGIGTSFNGV

SEQ ID NO: 11 - protein, 206 aa, Artificial Sequence
  Amino acids 105-310 of Cts1 (Coccidioides posadiasii, GenBank AAA92643.1), with C108N, K116R, P140A, T143N, K156H, D164E, G165A, E174D, D175E, E176D, F182W, K187H, K205R, G250V, K261R, S268T, K285R, I286V, A296L, and S206Tsubstitutions.
VYGNIKQMYLLRKNNRNLKTLLSIGGWTYSPNFKTAASNEEGRKKFADTSLHLMKDLGFEAIDIDWEYPDEDKQANDWVLLLHACREALDAYSAKHPNGKRFLLTIASPAGPQNYNKLKLAEMDKYLDFWNLMAYDFSGSWDKVSVHMSNVFPSTTRPESTPFTSDKAVKDYIKAGVPANRVVLGMPLYGRLFASTDGIGTTFNGV

SEQ ID NO: 12 - protein, 206 aa, Artificial Sequence
  Amino acids 105-310 of Cts1 (Coccidioides posadiasii, GenBank AAA92643.1), with K110R, K117H, L126V, K138R, E174D, D175E, L186V, R190K, A192V, K204H, K205R, A238I, S294T, K261R, S268T, D275E, A283L, G294A, G304I, and G309Msubstitutions.
VYGCIRQMYLLKHNNRNLKTLVSIGGWTYSPNFRTPASTEEGRKKFADTSLKLMKDLGFDGIDIDWEYPDEEKQANDFVLLVKACKEVLDAYSAKHPNGHRFLLTIASPAGPQNYNKLKLAEMDKYLDFWNLMIYDFSGSWDKVTGHMSNVFPSTTRPESTPFTSDKAVKEYIKAGVPLNKIVLGMPLYARAFASTDGIVTSFNMV